(19) United States
(12) Patent Application Publication
(10) Pub. No.: US 2019/0282322 A1
(43) Pub. Date: Sep. 19, 2019

(54) MEDICAL INSTRUMENTS WITH ADJUSTABLE OPTICAL FIBER

(71) Applicant: Alcon Inc., Fribourg (CH)

(72) Inventors: Alireza Mirsepassi, Irvine, CA (US); Kambiz Parto, Laguna Niguel, CA (US)

(21) Appl. No.: 16/299,876

(22) Filed: Mar. 12, 2019

Related U.S. Application Data
(60) Provisional application No. 62/642,755, filed on Mar. 14, 2018.

(51) Int. Cl. A61B 90/30; A61F 9/007; A61F 9/008; A61B 5/00

(52) U.S. Cl. CPC A61B 90/30; A61F 9/00736; A61B 2090/306; A61B 5/0066; A61F 9/00821

(57) ABSTRACT

An example illuminated microsurgical instrument comprises a handpiece, a tubular member connected to the handpiece to perform a medical procedure at an interventional site, a sheath member surrounding a portion of the tubular member and extending toward the distal tip of the tubular member, and an optical fiber positioned within the sheath member and connected to the sheath member, wherein a distal tip of the optical fiber is recessed within the sheath member and directed toward the distal tip of the tubular member. The sheath member and distal tip of the optical fiber may be movable between a proximal position at a first distance from the distal tip of the tubular member and a distal position at a second distance from the distal tip of the tubular member, wherein the second distance is shorter than the first distance. Movement of the sheath member moves the distal tip of the optical fiber to generate a wider illumination or a narrower illumination on the site being visualized.

### PRIORITY CLAIM

This application claims the benefit of priority of U.S. Provisional Patent Application Ser. No. 62/642,755 titled “Medical Instruments with Adjustable Optical Fiber,” filed on Mar. 14, 2018, whose inventors are Alireza Mirsepassi and Kambiz Parto, which is hereby incorporated by reference in its entirety as though fully and completely set forth herein.

### TECHNICAL FIELD

The present disclosure is directed to systems and instruments for use in medical procedures, and more particularly, to methods and systems involving a need for an optical fiber to be inserted within a body cavity.

### BACKGROUND

Medical procedures are often performed within significantly limited confines of a particular body structure or cavity, such as within the posterior chamber of the human eye. For example, vitreo-retinal procedures are commonly performed to treat many serious conditions of the posterior segment of the eye. In particular, vitreo-retinal procedures may treat conditions such as age-related macular degeneration (AMD), diabetic retinopathy and diabetic vitreous hemorrhage, macular hole, retinal detachment, epiretinal membrane, cytomegalovirus (CMV) retinitis, and many other ophthalmic conditions.

A surgeon performs vitreo-retinal procedures with a microscope and special lenses designed to provide a clear image of the posterior segment. Several tiny incisions just a millimeter or so in diameter are made on the sclera at the pars plana. The surgeon inserts microsurgical instruments through the incisions, such as a light source to illuminate inside the eye, an infusion line to maintain the eye's shape during surgery, and other instruments to cut and remove the vitreous body. A separate incision may be provided for each microsurgical instrument when using multiple instruments simultaneously.

During such procedures, proper illumination of the inside of the eye is important. Typically, an optical fiber is inserted into one of the incisions in the eye to provide the illumination. A light source, such as a halogen tungsten lamp or high pressure arc lamp (metal-halides, Xenon), may be used to produce the light carried by the optical fiber into the eye. In some embodiments, the light source may be a white light, single wavelength (e.g., green light centered at 532 nanometer wavelength), red+blue+green (RGB), or RGB plus additional wavelengths. The light passes through several optical elements (typically lenses, mirrors, and attenuators) and is transmitted to the optical fiber that carries the light into the eye.

In such procedures, incisions are typically only made large enough to accommodate the size of the microsurgical instrument being inserted into the interior of the eye. Efforts to minimize the incision size generally involve reducing the size of the microsurgical instrument. However, a reduction in size can result in a reduction in instrument strength or rigidity. Depending on the size of the microsurgical instrument employed, the incision may be small enough to render a resulting wound substantially self-healing, thereby eliminating the need to employ additional procedures to close the incision, such as sutures. Also, reducing the number of incisions may be accomplished by integrating various microsurgical instruments. For example, the optical fiber may be incorporated into the working end of a microsurgical instrument. Unfortunately, at least some prior attempts at integrating optical fibers with microsurgical instruments have resulted in a decrease in illuminating efficiency or in other visualization problems that otherwise adversely effected the distribution of light emitted from the optical fibers.

### SUMMARY

The present disclosure is directed to exemplary illuminated microsurgical instruments and associated methods.

In one example, an illuminated microsurgical instrument may comprise a handpiece; a distally projecting tubular member connected to the handpiece, the tubular member arranged to perform a medical procedure at an interventional site, the tubular member having a distal tip and an outer surface; a sheath member surrounding a portion of the tubular member and extending toward the distal tip of the tubular member; and an optical fiber positioned within the sheath member and connected to the sheath member, wherein a distal tip of the optical fiber is recessed within the sheath member and directed toward the distal tip of the tubular member. The sheath member and distal tip of the optical fiber may be movable between a proximal position at a first distance from the distal tip of the tubular member and a distal position at a second distance from the distal tip of the tubular member, wherein the second distance is shorter than the first distance.

The distal tip of the optical fiber may be fixed in position relative to a distal end of the sheath member. The instrument may further comprise an actuator connected to the sheath member to move the sheath member between the proximal position and the distal position.

In an example method of using an illuminated microsurgical instrument, a user moves the sheath member between a proximal position at a first distance from the distal tip of the tubular member and a distal position at a second distance from the distal tip of the tubular member, wherein the second distance is shorter than the first distance. The site being visualized may be, for example, the retina, an area within the posterior chamber distal to the instrument, or an area within the posterior chamber adjacent a port near the distal end of the instrument. Movement of the sheath member moves the distal tip of the optical fiber closer to or farther away from the site being visualized. When the sheath member is in the proximal position, the distal tip of the optical fiber is farther away from the site being visualized and thus illuminates a wider area of the site being visualized than when the sheath member is in the distal position. When the sheath member is in the distal position, the distal tip of the optical fiber is closer to the site being visualized and thus illuminates a narrower area of the site being visualized with higher beam intensity than when the sheath member is in the proximal position.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the accompanying drawings and the following detailed description.

### BRIEF DESCRIPTION OF THE DRAWINGS

The accompanying drawings illustrate implementations of the devices and methods disclosed herein and, together with the description, serve to explain the principles of the present disclosure.

FIG. 1 illustrates an example of an illuminated microsurgical instrument with a sheath member and optical fiber in a proximal position, according to an embodiment.

FIG. 2 illustrates the illuminated microsurgical instrument of FIG. 1 with the sheath member and optical fiber in a distal position, according to an embodiment.

FIG. 3 illustrates a close-up of the optical fiber recessed in the sheath, according to an embodiment.

The accompanying drawings may be better understood by reference to the following detailed description.

### DETAILED DESCRIPTION

The present disclosure contains subject matter that is related to the subject matter disclosed in U.S. Provisional Patent Application No. 62/423,499, filed Nov. 17, 2016 (entitled “Medical Instrument with an Integrated Optical Fiber”), U.S. Provisional Patent Application No. 62/543,548 filed Aug. 10, 2017 (entitled “Medical Instrument with an Integrated Optical Fiber”), U.S. Non-Provisional patent application Ser. No. 15/805,519 filed Nov. 7, 2017 (entitled “Medical Instrument with an Integrated Optical Fiber”) and U.S. Non-Provisional patent application Ser. No. 15/814,929 filed Nov. 16, 2017 (entitled “Medical Instrument with an Integrated Optical Fiber”), the disclosures of which are hereby incorporated by reference in their entirety as though fully and completely set forth herein.

For the purposes of promoting an understanding of the principles of the present disclosure, reference will now be made to the implementations illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure. For simplicity, in some instances the same reference numbers are used throughout the drawings to refer to the same or like parts.

The present disclosure is broadly directed to systems and instruments for providing an optical fiber within a body cavity during an operation performed therein without requiring a separate incision to be made. More particularly, some aspects of the present disclosure are directed to systems and instruments for providing for illumination through an optical fiber positioned within the body cavity. In some examples, the illumination is provided through an optical fiber extending along a length of another surgical instrument or tool within the body cavity. For example, a vitrectomy procedure may be performed to remove vitreous from the eye of a patient using a vitrectomy probe introduced into the eye to position a vitrectomy needle at an interventional site. Rather than form two incisions in the eye of the patient, the optical fiber may be positioned along a portion of the vitrectomy needle. The optical fiber may have a distal tip through which light is introduced or emitted into the posterior chamber of the eye, when the distal tip of the vitrectomy probe is positioned within the eye. The removal of the vitreous may be of particular importance, because residual vitreous can cause post-operative retinal tearing, retinal detachment, etc.

The clear vitreous may be visualized due to light scattering off the vitreous fibers contained within it. The lighting may be directed proximate the cutting portion of the vitrectomy probe in order to better visualize the vitreous being cut. Depending on the implementation, the optical fiber may be secured at least partially to a sheath that also protects the optical fiber. Thus, implementations of the present disclosure provide for improved illumination for inner-cavity procedures, such as vitrectomy procedures, while minimizing the number of incisions required to be made to permit entry to the cavity. The illumination provided by implementations of the present disclosure may result in high irradiance at the surgical site, e.g., at the port of the vitrectomy needle. This may provide for a high signal to noise ratio or contrast to facilitate visualization of the fibers in the vitreous. While specific examples of implementations are provided herein that are directed to vitrectomy procedures and devices, the principles of the present disclosure extend beyond vitrectomy instruments and procedures.

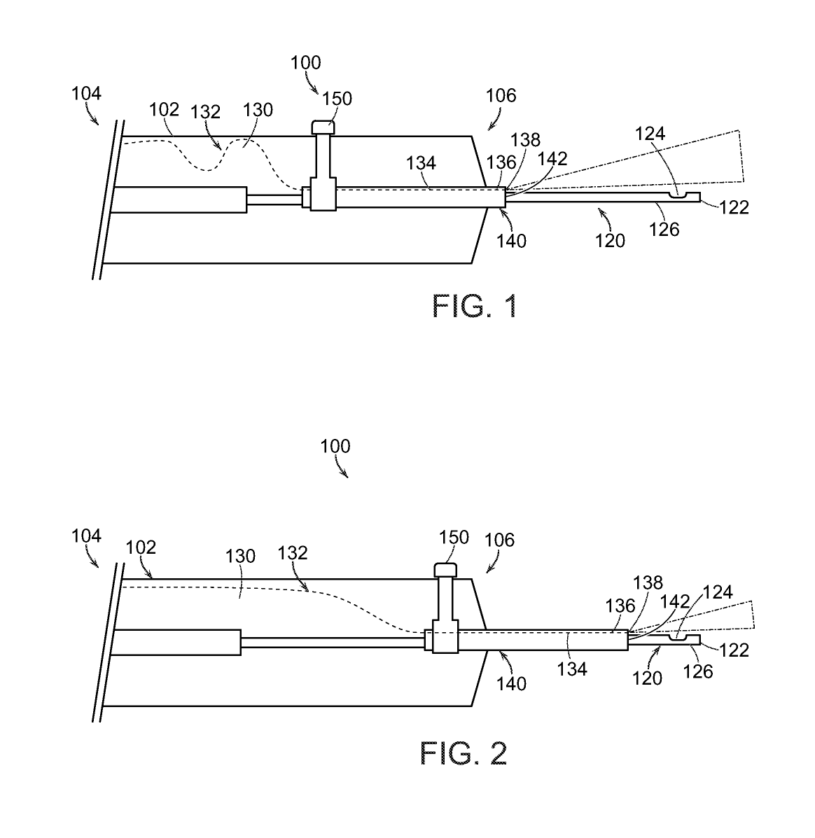

FIG. 1 illustrates an example of an illuminated microsurgical instrument. In one example, the instrument 100 may be a vitrectomy probe configured to be held in the hand of a surgeon during use. The instrument or probe 100 includes a handpiece 102 having a proximal end 104 and a distal end 106.

An elongate tubular member 120 extends from the distal end 106 of the handpiece 102. This distally projecting tubular member has an outer surface 126 and terminates in a distal tip 122 at its distal end. The tubular member 120 may be a vitrectomy needle that includes inner and outer components that may be used for cutting vitreous proximate a distal tip 122 of vitrectomy needle 120 during vitrectomy procedures. The tubular member 120 may include an opening or port 124 proximate its distal end 122. For example, when the instrument 100 is a vitrectomy probe, the opening or port 124 may be an opening or port into which vitreous may be aspirated and cut during a vitrectomy procedure.

In one example, an activation energy source provides an oscillation energy to components of the probe 100, in order to provide an oscillatory motion to the inner component of the vitrectomy needle 120. For example, a pneumatic source may be coupled to an oscillation motor, which in turn drives the inner component of the vitrectomy needle 120 in an oscillatory motion. In other examples, the oscillation motion of the inner component of the vitrectomy needle 120 may be provided by an oscillating electric motor or other non-pneumatic activation means.

The probe 100 may also be coupled to an aspiration source to enable aspiration of material through the probe 100. For example, the inner component of the vitrectomy needle 120 may have a lumen extending therethrough such that material may be aspirated from the site of the vitrectomy procedure. For example, as seen in FIG. 3, inner tubular member 343 is an elongate tubular member extending within a lumen 347 of the elongate tubular member 120. The distal edges 339 of the inner tubular member 343 may be sharpened or include a shape to facilitate cutting of vitreous as the inner tubular member 343 oscillates back and forth within the lumen 347 and cycles past the port 124. Vitreous aspirated into the port 124 may be cut by the oscillating inner tubular member 343.

The handpiece 102 includes a chamber 130 inside the handpiece 102. A length of an optical fiber 132 extends within the chamber 130. The optical fiber 132 may be connected at its proximal end to a control system (not shown) and at its distal end 136 to a sheath member or sleeve 140 which is around the tubular member 120. The chamber 130 may include sufficient space to accommodate slack of the optical fiber 132 when it is in the proximal position, as described in more detail below.

The sheath member or sleeve 140 surrounds a portion of the tubular member 120 and extends toward the distal tip 122 of the tubular member 120 along the outer surface of the tubular member 120. The tubular member 120 extends beyond a distal end 142 of the sheath member or sleeve 140. The sheath member or sleeve 140 is capable of sliding axially along the tubular member or needle 120. In order to prevent backflow, a flexible sealing element may be provided on the inner surface of the sheath member or sleeve 140 to create a seal with the tubular member or needle 120. The sealing element may be capable of sliding along the tubular member or needle 120 while maintaining a seal with the tubular member or needle 120.

The optical fiber 132 extends within the sheath member or sleeve 140 such that the sheath member or sleeve 140 surrounds and encloses a distal section 134 of the optical fiber 132. The optical fiber 132 may be connected at its distal end 136 to the sheath member or sleeve 140. For example, the distal end 136 of the optical fiber 132 may be affixed to the inner surface of the sheath member or sleeve 140, for example by adhesive 358. Thus, the distal tip 138 of the optical fiber 132 is fixed in position relative to the distal end 142 of the sheath member or sleeve 140. The distal tip 138 of the optical fiber 132 is directed toward the distal tip 122 of the tubular member 120 and toward the opening or port 124.

The distal tip 138 of the optical fiber 132 may be connected to the inner surface of the sheath member or sleeve 140 such that it is recessed by a small distance D2 from the distal end 142 of the sheath member or sleeve 140 (e.g., see FIG. 3). For example, the distal tip 138 of the optical fiber 132 may be recessed from the distal end 142 of the sheath member or sleeve 140 by a distance D2 ranging from about 10 μm (micrometers) to about 50 μm. In some implementations, the distance D2 may be about 25 μm. Having the distal tip 138 of the optical fiber 132 recessed from the distal end 142 of the sheath member or sleeve 140 can help to protect the optical fiber 132 and can help to provide the desired illumination pattern while minimizing glare or any bright spot from the distal tip 138 of the optical fiber 132. The recessed position of the optical fiber 132 inside the sheath may also be safer once the sheath/optical fiber is inside the eye.

In order to generate the desired illumination pattern, the distal tip 138 of the optical fiber 132 may have an angled face that is angled toward the outer surface 126 of the tubular member 120. The angled face of the distal tip 138 of the optical fiber 132 causes a field of illumination to be directed substantially away from the outer surface 126 of the tubular member 120. The angled face of the distal tip 138 of the optical fiber 132 may form an angle with respect to the outer surface 126 of the tubular member 120 ranging from about 30 degrees to about 40 degrees.

The sheath 140 further surrounds and encloses the optical fiber 132. The optical fiber 132 includes a face at the distal end thereof. Illumination in an illumination beam 354 may be emitted from the face to illuminate an area proximate the port 124. For example, during a vitrectomy procedure, the illumination beam 354 may be generally ovoid in shape and centered at the central illumination point 356, as shown in FIG. 3. The illumination beam 354 may span an angle A1 and may have a portion that is tangential to the outer surface 126 of the elongate tubular member 120. In some implementations of the probe, the face may be angled such that no portion of the illumination beam 354 contacts the outer surface of the elongate tubular member 120 at all. The face may be a beveled face that forms an angle, which may range from about 20° to about 50°. In some implementations, the angle is about 35°. Other angles are contemplated in other implementations.

As noted above, to protect the face at the distal end of the optical fiber 132, the distal end thereof may be offset from the distal edge 142 of the sheath 140 by a distance D2. This distance D2 may provide sufficient protection of the optical fiber 132 and the face and may also provide a limit to the angle A1 of the illumination beam 354 to control the light and better enable the surgeon to visualize tissue material proximate the distal tip 122, thereby aiding a surgeon in removing vitreous via the port 124. A central illumination point 356 may be angled away from the surface of the outer tubular member 120 to avoid glare being reflected off the exterior surface. In some implementations, some rays of the illumination beam may be incident upon the exterior of the outer tubular member 120.

Because the distal end 138 of the optical fiber 132 is affixed to the inner surface of the sheath member or sleeve 140, when the sheath member or sleeve 140 is moved axially along the tubular member or needle 120, the distal section of the optical fiber 132 moves with it. While FIG. 3 shows the optical fiber 132 affixed to the sleeve 140 in a position closer to the needle 120 than the sleeve 140 (e.g., with more adhesive between the optical fiber 132 and the sleeve 140 than between the optical fiber 132 and the sleeve 140), in some embodiments, the optical fiber 132 may be affixed to the sleeve 140 in a position closer to the sleeve 140 than the needle 120.

A distal edge 142 of the sheath 140 may be offset from a center of the port 124 by a distance D1. The distance D1 may be varied by moving the sheath member or sleeve 140. In some embodiments, D1 may range from about 2 mm to about 3 mm. Other implementations may have a distance D1 that is greater or lesser than this range (e.g., moved between 2 mm and 30 mm as further described below). In order to move the sheath member or sleeve 140 and the distal section of the optical fiber 132 axially along the tubular member or needle 120 (to vary D1), the instrument 100 may further comprise an actuator. The actuator may be, for example, a mechanical actuator or an electrical actuator. In one example, shown in FIG. 1, the actuator comprises a button 150 that is connected to the sheath member or sleeve 140. The button may be slidable along a track in the handpiece 102. A user of the instrument, e.g., the surgeon, may move the button in order to advance and retract the sheath member or sleeve 140 axially along the tubular member or needle 120. In other examples, the actuator may be a solenoid or other electrically-activated actuator that is connected to a control system. The user of the instrument may activate the actuator via the control system, for example by a foot pedal, remote button or dial, or other means, in order to advance and retract the sheath member or sleeve 140 axially along the tubular member or needle 120.

FIG. 1 illustrates the illuminated microsurgical instrument 100 with the sheath member or sleeve 140 and the optical fiber 132 in a proximal position. In this position, the distal end 142 of the sheath member or sleeve 140, and the distal tip 138 of the optical fiber 132, are at a first distance from the distal end 122 and the port or opening 124 of the tubular member or needle 120. This first distance may be, for example, about 15 mm to about 30 mm in some implementations. During a procedure, the site being visualized may be, for example, the retina, an area within the posterior chamber distal to the tubular member or needle 120, or an area within the posterior chamber adjacent the port or opening 124 of the tubular member or needle 120. In this proximal position, because of the greater distance from the site being visualized, the optical fiber 132 illuminates a relatively wider area of the site being visualized.

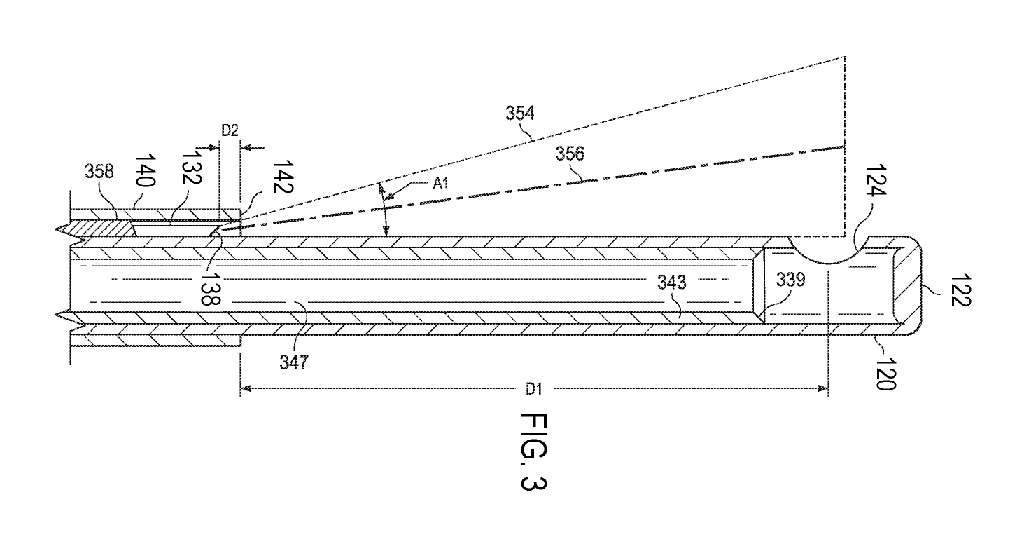

FIG. 2 illustrates the illuminated microsurgical instrument 100 with the sheath member or sleeve 140 and the optical fiber 132 in a distal position. In this position, the distal end 142 of the sheath member or sleeve 140, and the distal tip 138 of the optical fiber 132, are at a second distance from the distal end 122 and the port or opening 124 of the tubular member or needle 120, the second distance being shorter than the first distance. This second distance may be, for example, about 2 mm to about 5 mm in some implementations. In this distal position, because of the closer distance to the site being visualized, the optical fiber 132 illuminates a relatively narrower area of the site being visualized, with higher beam intensity.

As can be appreciated from the above description, the sheath member or sleeve 140 and the distal tip 138 of the optical fiber 132 are movable between a proximal position at a first distance from the distal tip 122 of the tubular member 120 and a distal position at a second distance from the distal tip 122 of the tubular member 120, wherein the second distance is shorter than the first distance. The sheath member or sleeve 140 and the distal tip 138 of the optical fiber 132 may be moved by an actuator as described above. The movement may be stopped at the first position, the second position, or any desired position in between in order the effect the desired illumination. When the distal tip 138 of the optical fiber 132 is farther away from the site being visualized, it projects a wider illumination onto the site being visualized, and when it is closer to the site, it projects a narrower, more intense illumination onto the site being visualized.

In an example method of use of the instrument 100, the user (e.g., surgeon) positioning the instrument 100 into a desired position for a microsurgical procedure. As mentioned above, the site being visualized may be, for example, the retina, an area within the posterior chamber distal to the tubular member or needle 120, or an area within the posterior chamber adjacent the port or opening 124 of the tubular member or needle 120. In order to obtain the desired visualization, i.e., either a wider illumination or a narrower illumination on the site being visualized, the user moves the sheath member 140 between a proximal position at a first distance from the distal tip 122 of the tubular member 120 and a distal position at a second distance from the distal tip 122 of the tubular member 120, wherein the second distance is shorter than the first distance. For example, a wider illumination area, with the sheath member at or close to the proximal position, may be advantageous for situational awareness or to visualize the retina. A narrower illumination area, with the sheath member at or close to the distal position, may be advantageous for an intense beam to visualize the vitreous near the distal tip 122 of the tubular member 120 during a vitrectomy procedure.

In one example implementation, dimensions may be as follows. The optical fiber 132 may have a diameter of about 20 μm to about 40 μm, or more particularly a diameter of 30 μm, although other sizes may be used. In some embodiments, the optical fiber may be a high intensity nanofiber that allows increased illumination (e.g., the nanofiber may be made of, for example, glass and transfer more light then prior, mainly plastic optical fibers). The tubular member may be, for example, 27 gauge, having an outer diameter of about 400 μm, although larger and smaller sizes may be used. The sheath member is sized to have an inner diameter large enough to accommodate the tubular member and the optical fiber, with possible clearance. The sheath member may have a wall thickness of about 20 μm to about 25 μm, although other sizes may be used. These dimensions are only to give a possible example, as dimensions may be varied within the scope of the disclosure.

As noted herein, some of the more specific implementations are described with respect to a vitrectomy probe in which an optical fiber provides for illumination of the vitreous at the distal tip of the vitrectomy probe. It should be noted that the described optical fiber may provide for other functions in other implementations. For example, the optical fiber included in implementations of the instrument may provide for transmission of laser light to provide a photocoagulation laser at a distal tip of the instrument. Additionally, the instrument may be a non-surgical medical instrument in other implementations. For example, additional implementations may utilize the optical fiber in the performance of optical coherence tomography (OCT) imaging, rather than or in addition to any surgical functions performed by implementations of the medical instrument. Accordingly, such instruments are included within the scope of the present disclosure.

Persons of ordinary skill in the art will appreciate that the implementations encompassed by the present disclosure are not limited to the particular exemplary implementations described above. In that regard, although illustrative implementations have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An illuminated microsurgical instrument comprising:
a handpiece;
a distally projecting tubular member connected to the handpiece, the tubular member arranged to perform a medical procedure at an interventional site, the tubular member having a distal tip and an outer surface;
a sheath member surrounding a portion of the tubular member and extending toward the distal tip of the tubular member; and
an optical fiber positioned within the sheath member and connected to the sheath member, wherein a distal tip of the optical fiber is recessed within the sheath member and directed toward the distal tip of the tubular member;
wherein the sheath member is movable between a proximal position at a first distance from the distal tip of the tubular member and a distal position at a second distance from the distal tip of the tubular member, wherein the second distance is shorter than the first distance.

2. The illuminated microsurgical instrument of claim 1, wherein the distal tip of the optical fiber is fixed in position relative to a distal end of the sheath member.

3. The illuminated microsurgical instrument of claim 1, further comprising an actuator connected to the sheath member to move the sheath member between the proximal position and the distal position.

4. The illuminated microsurgical instrument of claim 3, wherein the actuator is a mechanical actuator.

5. The illuminated microsurgical instrument of claim 3, wherein the actuator is an electrical actuator.

6. The illuminated microsurgical instrument of claim 5, wherein the electrical actuator is at least partially controlled through a footswitch.

7. The illuminated microsurgical instrument of claim 1, wherein the distal tip of the optical fiber has an angled face that is angled toward the outer surface of the tubular member.

8. The illuminated microsurgical instrument of claim 7, wherein the angled face of the distal tip of the optical fiber causes a field of illumination to be directed substantially away from the outer surface of the tubular member.

9. The illuminated microsurgical instrument of claim 7, wherein the angled face of the distal tip of the optical fiber forms an angle with respect to the outer surface of the tubular member ranging from about 30 degrees to about 40 degrees.

10. The illuminated microsurgical instrument of claim 1, wherein the illuminated microsurgical instrument is a vitrectomy probe.

11. A method of using an illuminated microsurgical instrument, comprising:
positioning the illuminated microsurgical instrument into a desired position for a microsurgical procedure, the illuminated microsurgical instrument comprising a handpiece; a distally projecting tubular member connected to the handpiece, the tubular member arranged to perform a medical procedure at an interventional site, the tubular member having a distal tip and an outer surface; a sheath member surrounding a portion of the tubular member and extending toward the distal tip of the tubular member; and an optical fiber positioned within the sheath member and connected to the sheath member, wherein a distal tip of the optical fiber is recessed within the sheath member and directed toward the distal tip of the tubular member; and
moving the sheath member between a proximal position at a first distance from the distal tip of the tubular member and a distal position at a second distance from the distal tip of the tubular member, wherein the second distance is shorter than the first distance.

12. The method claim 11, wherein the distal tip of the optical fiber is fixed in position relative to a distal end of the sheath member.

13. The method of claim 11, further comprising an actuator connected to the sheath member to move the sheath member between the proximal position and the distal position.

14. The method of claim 13, wherein the actuator is a mechanical actuator.

15. The method of claim 13, wherein the actuator is an electrical actuator.

16. The method of claim 15, wherein the electrical actuator is at least partially controlled through a footswitch.

17. The method of claim 11, wherein the distal tip of the optical fiber has an angled face that is angled toward the outer surface of the tubular member.

18. The method of claim 17, wherein the angled face of the distal tip of the optical fiber causes a field of illumination to be directed substantially away from the outer surface of the tubular member.

19. The method of claim 17, wherein the angled face of the distal tip of the optical fiber forms an angle with respect to the outer surface of the tubular member ranging from about 30 degrees to about 40 degrees.

20. The method of claim 11, wherein the illuminated microsurgical instrument is a vitrectomy probe.
